(19) United States
(12) Patent Application Publication
(10) Pub. No.: US 2022/0218801 A1
(43) Pub. Date: Jul. 14, 2022

(54) COMBINATION GMCI AND ATRI CANCER TREATMENT

(71) Applicants: Candel Therapeutics, Inc., Needham, MA (US); Brigham and Women's Hospital, Inc., Boston, MA (US)

(72) Inventors: Carlos Estuardo Aguilar-Cordova, Waban, MA (US); Guzik Brian, Arlington, MA (US); Marilin Koch, Boston, MA (US); Sean Lawler, Scituate, MA (US)

(21) Appl. No.: 17/709,009

(22) Filed: Mar. 30, 2022

Related U.S. Application Data
(63) Continuation of application No. PCT/US2020/053335, filed on Sep. 29, 2020.
(60) Provisional application No. 62/908,209, filed on Sep. 30, 2019.

(51) Int. Cl. A61K 38/50; A61K 38/45; A61K 31/522; A61K 31/513; A61K 31/5377; A61K 31/36; A61K 31/505; A61K 31/4745; A61K 31/519; A61K 31/497; A61K 31/4965; A61K 45/06; A61P 35/00

(52) U.S. Cl. CPC A61K 38/50; A61K 38/45; A61K 31/522; A61K 31/513; A61K 31/5377; A61K 31/36; A61P 35/00; A61K 31/4745; A61K 31/519; A61K 31/497; A61K 31/4965; A61K 45/06; A61K 31/505

(57) ABSTRACT

Disclosed are methods of treating a cancer in a subject, comprising treating the subject with a combination of gene-mediated cytotoxic immunotherapy and a DNA damage response inhibitor.

### CROSS REFERENCE TO RELATED APPLICATIONS

This application is a continuation of International Patent Application No. PCT/US2020/053335, filed on Sep. 29, 2020, which claims priority to U.S. Provisional Application No. 62/908,209, filed on Sep. 30, 2020, the contents of which are hereby incorporated by reference in their entirety.

### FIELD OF THE INVENTION

This disclosure is in the fields of molecular biology, immunology, and cancer therapy.

### BACKGROUND

Cancer afflicts about 17 million people yearly worldwide. Commonly used methods of treating cancer include surgical resection, radiation therapy, chemotherapy, immunotherapy, oncolytic viral therapy, and combinations thereof.

More recently, cancer has been treated with gene-mediated cytotoxic immunotherapy (GMCI). GMCI utilizes a viral vector to deliver a gene which, when delivered to a target tissue and expressed, can activate a separately delivered prodrug that ultimately causes cytotoxicity and cell death due to resulting defects in the targeted cell's DNA repair mechanism

A component of GMCI activity is stimulation of the treated subject's immune response against the tumor cells. This mechanism of action involves activation and activation mitotic division of the patients' immune cells in situ. However, cancer therapy using this modality is not always efficacious. For example, although studies have indicated that GMCI has some activity in brain cancer patients, improvements in commonly measured outcomes such as survival or tumor shrinkage have not been in all patient and are infrequently durable.

Another type of cancer therapy involves the administration of a DNA damage repair inhibitor such as an inhibitor of ataxia telangiectasia and rad3-related (ATR) kinase. ATR kinase is a serine/threonine protein kinase upregulated in a variety of cancer cell types. It plays a key role in DNA repair, cell cycle progression, and survival and is activated by DNA damage caused during DNA replication-associated stress. ATRi's selectively bind to, and inhibit, ATR kinase activity and prevent ATR-mediated signaling in the ATR-checkpoint kinase 1 (Chk1) signaling pathway. This prevents DNA damage checkpoint activation, disrupts DNA damage repair, and induces tumor cell apoptosis. Representative, nonlimiting ATR inhibitors include: BAY1895344, Schisandrin B, NU6027, NVP-BEZ235, VX-803, VX-970(M6620), VE-821, VE-822, AZ20, and AZD6738

Unfortunately, ATRi's may have toxicities, and are unlikely to have efficacy in all patients or tumors within a patient, including tumor responses that are not durable (Weber and Ryan 2015; Minchom et al. 2018)). ATR function is known to be essential for normal function of cells and tissues, and there are toxicities observed with inhibition of ATR function. Even only partial loss of ATR function may lead to an increased frequency of breakage at fragile sites of chromosomes, which may damage tissue in which cell proliferation is needed for maintenance (Ruzankina 2007). Partial loss of ATR function in mice was also observed to increase DNA replication stress, and lead to accelerated aging and decreased lifespan (Murga 2010). Full loss of ATR function in mice has been associated with embryonic lethality (Brown and Baltimore 2000). Thus, the disruption of ATR function has the potential for severe side effects due to toxicity in normally proliferative tissues, particularly in patients that are treated with ATRi drugs in combination with chemotherapies that may cause DNA replication stress such as temozolomide, including symptoms such as thrombocytopenia and/or neutropenia (Weber and Ryan 2015).

While GMCI and ATRi's each have anti-tumor activity, improvements in their use are desired to provide better clinical outcomes such as improves survival, increased times to disease progression, increase frequency of tumor responses, increased durability of responses, increased tumor cell killing, enhanced immune activity against tumor cells, decreased toxicities, decreased dosages of the drugs required for efficacy, improved dosing schedules of the drugs.

### SUMMARY

It has been discovered that treatment of a cancer in a subject with a combination of GMCI and an ATR inhibitor has synergistically greater effects (i.e., greater than the effects of each added together) than the effects provided by either GMCI or ATR inhibition treatment, alone. For example, the cytotoxicity delivered from treating a cancer with a combination of GMCI and an ATR inhibitor is unexpectedly greater compared to the cytotoxicity delivered when treating the cancer with GMCI or an ATR inhibitor, alone (or greater than the cytotoxicity of both added together). In addition, it was discovered that the combination therapy results in more rapid killing of cancer cells and more rapid tumor shrinkage than was found when either therapy, alone, was used. Also, tumor responses to treatment more frequently occur in patients treated with the combination, than in patients treated with only GMCI or an ATR inhibitor. In addition, tumor responses that occur in patients treated the combination of GMCI and an ATR inhibitor are more frequent, of greater magnitude, and are more durable than those treated with GMCI or an ATR inhibitor, alone.

These discoveries have been exploited to provide the present disclosure, which in part provides a method of treating cancer in a subject, comprising treating the subject with a combination of gene-mediated cytotoxic immunotherapy and an ATR inhibitor which is a DDRI.

In one aspect, the disclosure provides a method of treating a cancer in a subject, comprising treating the subject with a combination of gene-mediated cytotoxic immunotherapy and an ATR inhibitor which is a DDRI.

In some embodiments, GMCI comprises: administering a viral vector encoding thymidine kinase or cytosine deaminase to the mammal with a tumor or to a tumor resection site in the mammal; and administering a prodrug to the mammal, the prodrug being activated by thymidine kinase or cytosine deaminase.

In certain embodiments, the vector is an adenovirus, an adeno-associated virus (AAV), a lentivirus, a retrovirus, a herpes virus, a New Castle Disease Virus, a coxsackievirus, or a vaccinia virus. In some embodiments, the vector is replication-incompetent or replication deficient.

In some embodiments, the prodrug comprises ganciclovir, acyclovir, valacyclovir, valgancyclovir, famiciclovir, or an analog thereof. In certain embodiments, the prodrug comprises de 5-flurocytosine or an analog thereof.

In some embodiments, the ATRi administration is before, during, or after GMCI administration. In some embodiments, the ATRi is an inhibitor of ataxia-telangiectasia mutated kinase and Rad3-related kinase. In particular embodiments, the ATR inhibitor is BAY1895344, Schisandrin B, NU6027, NVP-BEZ235, VX-803, VX-970(M6620), VE-821, VE-822, AZ20, or AZD6738.

In certain embodiments, the method further comprising administering radiotherapy and/or chemotherapy to, and/or performing surgery on, the mammal before, during, or following GMCI and/or administering the ATR inhibitor.

In another aspect, the disclosure provides use of combination of gene-mediated cytotoxic immunotherapy and an ATR inhibitor which is a DDRI for the treatment of a cancer in a subject.

### BRIEF DESCRIPTION OF THE DRAWING

The foregoing and other objects of the present disclosure, the various features thereof, as well as the disclosure itself may be more fully understood from the following description, when read together with the accompanying drawings in which:

FIG. 1 is a schematic representation of the method of enhanced cancer cell death associated with the treatment of GMCI and ATR inhibition;

is a graphic representation showing reduced glioma cell viability after treatment with GMCI, ATR inhibition, and combination of GMCI and ATR inhibition;

FIG. 2 is a schematic representation of a study examining the frequency of double-stranded DNA breaks in glioma cells treated with GMCI, ATR inhibition, or the combination;

FIG. 3 is a table showing the results of a study examining the frequency of double-stranded DNA breaks in glioma cell treated the GMCI, ATR inhibition, or the combination;

FIG. 4 is a schematic representation of a study examining double-stranded DNA breaks in glioma cells treated with GMCI, ATR inhibition, or the combination thereof; and

FIG. 5A-5D are representations of fluorograms of untreated glioma cells (FIG. 5A); GMCI-treated glioma cells (FIG. 5B); glioma cells treated with IC₅₀ AZD6738 (FIG. 5C); or glioma cells treated with GMCI and AZD6738 (FIG. 5D), and then stained with antibody for H2AX(P-Ser139) as a marker for double-stranded breaks.

### DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the term “administration” of an agent or drug to a subject includes any route of introducing or delivering the agent to a subject to perform its intended function. Administration can be carried out by any suitable route, including, but not limited to, orally, intratumorally, intracranially, parenterally (intravenously, intramuscularly, intraperitoneally, or subcutaneously), rectally, or topically. Administration includes self-administration and the administration by another.

The terms “ATRi” and “ATR inhibitors” are used herein to encompass inhibitors of ataxia-telangiectasia mutated kinase and Rad3-related kinase

As used herein, the term “cancer” refers to a class of diseases of humans and animals characterized by uncontrolled cellular growth. “Cancer” is used interchangeably with the terms “tumor,” “malignancy”, “hyperproliferation” and “neoplasm(s). The term “cancer cell(s)” is interchangeable with the terms “tumor cell(s),” “malignant cell(s),” “hyperproliferative cell(s),” and “neoplastic cell(s)” unless otherwise explicitly indicated. Similarly, the terms “hyperproliferative,” “hyperplastic,” “malignant” and “neoplastic” are used interchangeably, and refer to those cells in an abnormal state or condition characterized by rapid proliferation. Collectively, these terms are meant to include all types of hyperproliferative growth, hyperplastic growth, neoplastic growth, cancerous growths or oncogenic processes, metastatic tissues or malignantly transformed cells, tissues, or organs, irrespective of histopathologic type or stage of invasiveness.

As used herein, the term “effective amount” or “pharmaceutically effective amount” or “therapeutically effective amount” or “prophylactically effective amount” of a composition, is a quantity sufficient to achieve a desired therapeutic and/or prophylactic effect, e.g., an amount which results in the prevention of, or a decrease in, the symptoms associated with a disease that is being treated, e.g., a cancer. The amount of a composition administered to the subject will depend on the type and severity of the disease and on the characteristics of the individual, such as general health, age, sex, body weight and tolerance to drugs. It will also depend on the degree, severity and type of disease. In some embodiments, an effective amount of an oncolytic virus may be administered to a subject having cancer in an amount sufficient to exert oncolytic activity, causing attenuation or inhibition of tumor cell proliferation leading to primary and/or metastatic tumor regression.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, etc.

As used herein, the term “subject” refers to an organism administered one or more active agents. Typically, the subject is a mammal, such as an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like). Typically, the terms “subject” and “patient” are used interchangeably herein in reference to a human subject.

As used herein, the terms “treating” or “treatment” or “alleviation” refers to both therapeutic treatment and prophylactic or preventative measures, wherein the object is to prevent or slow down (lessen) the targeted pathologic condition or disorder. For example, a subject is successfully “treated” for a cancer, if after receiving a therapeutic amount of the compositions described herein, the subject shows observable and/or measurable reduction in or absence of one or more signs and symptoms of the cancer, e.g., reduction in the number of cancer cells or absence of the cancer cells; reduction in the tumor size; inhibition of tumor metastasis; inhibition, to some extent, of tumor growth; increase in length of remission, and/or relief to some extent, of one or more of the symptoms associated with the specific cancer; reduced morbidity and mortality and improvement of life issues.

The present disclosure relates, in part, to a method of killing a cancer or tumor cells, and thus treating cancer by using a combination of therapies comprising gene-mediated cytotoxic immunotherapy and ATR inhibitors.

### 1. GMCI

GMCI involves the administration of a viral vector carrying a gene encoding a cytotoxic protein to a subject afflicted with a cancer. When the viral vector is delivered to a target tissue in the subject, it expresses the gene, which, when in contact with a separately administered prodrug, activates the prodrug. The activated prodrug is cytotoxic to the cell and ultimately causes cell death due to resulting defects in the targeted cell's DNA repair mechanism.

A. Viral Vectors

Useful viral vectors include any viruses that can target a tissue and can carry a gene encoding a protein that can activate a prodrug. The viral vectors may be a virus that can replicate in the target tissue, or can be replication incompetent. Useful viral vectors include, but are not limited to, adenovirus, adeno-associated virus (AAV), lentivirus, retrovirus, herpes virus, New Castle Disease Virus, coxsackievirus, and vaccinia virus.

Useful genes to be carried by the viral vector are those genes which encode a “cytotoxic protein” that when expressed, is able to activate a prodrug, thereby causing a cytotoxic response. As used herein, “activates” means causes the prodrug to become cytotoxic. For example, activation of the prodrug may involve phosphorylation of the prodrug or its metabolite leading to the formation of a nucleotide analog that inhibits DNA repair by preventing DNA chain extension and DNA polymerase activity. This results in defective DNA repair in and around the targeted cancer cells, leading to cytotoxicity and ultimately, to cell death. Representative useful cytotoxic proteins include, but are not limited to, thymidine kinase and cytosine deaminase.

B. Prodrugs

Useful prodrugs include any that are in inactive form until they are contacted and activated by, the cytotoxic protein. Useful prodrugs administered in conjunction with thymidine kinase expressing vectors include, but are not limited to, ganciclovir, acyclovir, valacyclovir, valgancyclovir, famiciclovir, and analogs thereof. Useful prodrugs administered in conjunction with cytosine deaminase expressing vectors include 5-Flurocytosine.

### II. ATR Inhibitors

Cancer cells may have weakened DNA repair and DNA-damage signaling capabilities compared to normal cells, and may be more susceptible to DDR inhibition than are normal cells. Because the key regulators within repair mechanisms, such as use either ATP or nicotinamide adenine dinucleotide for their enzymatic: functions, they are readily accessible to small molecule inhibition at their catalytic sites. For example, ataxia-telangiectasia mutated kinase and Rad3-related (ATR) kinase are useful target kinases involved in the detection, signaling, and repair of double-stranded (Smith et al. (2003) Handbook of Cell Signaling); (O'Connor et al. 2007; Abraham 2004; Berghoff et al. 2014). Representative, nonlimiting ATR inhibitors include BAY1895344, Schisandrin B, NU6027, NVP-BEZ235, VX-803, VX-970(M6620), VE-821, VE-822, AZ20, and AZD6738 (Weber and Ryan 2015)) These inhibitors can be synthesized or can be commercially obtained.

### III. Combination Treatment

Treatment of a cancer in a subject according to the disclosure comprises a combination of GMCI (administration of a viral vector encoding a cytotoxic protein and administration of a prodrug activated by the cytotoxic protein) and ATRi administration.

Pharmaceutical formulations for this combination therapy comprise the viral vector, the prodrug, and/or an ATR inhibitor. These formulations are prepared using a pharmaceutically acceptable carrier or diluent (Nyberg-Hoffman and Aguilar-Cordova 1999) which does not affect the activity of the viral vector, prodrug, or DDRI. Pharmaceutically acceptable carriers or diluents are nontoxic to recipients at the dosages and concentrations employed. Representative examples of carriers or diluents for injectable solutions include water, isotonic saline solutions which may be buffered at a physiological pH or a pH for vector stability (such as phosphate-buffered saline or Tris-buffered saline), mannitol, dextrose, sucrose, glycerol, and ethanol, as well as polypeptides or proteins such as human serum albumin. The formulations may be prepared either as a liquid solution, or in solid form (e.g., lyophilized) which is suspended in a solution prior to administration. The carriers and/or diluents in the formulations are suitable for surface administration, injection, oral, or rectal administration.

A. GMCI Administration

Formulated viral vectors or viral particles may be administered to a wide variety of tissue and/or cell types where cancerous lesions may exist, including for example, the brain and/or spinal cord, bone marrow, eyes, the liver, nose, throat and lung, heart and blood vessels, spleen, skin, circulation, muscles, prostate, breast, pancreas, kidney, cervix, prostate, and other organs.

Various methods may be utilized within the context of the present disclosure in order to administer the viral vector or viral particles to the tumor. Vectors may be administered either directly (e.g., intravenously, intramuscularly, intraperitoneally, intra-lesionally, intra-cavitally, subcutaneously, or intravesically, during surgical intervention) or indirectly (e.g., orally, rectally, intraocularly, intranasally,) to the site of a tumor lesion. Alternatively, other clinically acceptable means of administration may be used, such as by various forms of catheter that can be introduced into the patient with minimal discomfort, followed by injection or release of the vector in conjunction with operations made possible by the catheter, such as multiple injection, introduction of radioactive seeds, tissue disruption and other means known to those skilled in the art. In addition, the viral vector may be delivered after formulation by various physical methods such as lipofection (Feigner et al. (1989) PNAS 84:7413-7417, direct DNA injection (Fung et al. (1983) PNAS 80:353-357; Seeger et al., PNAS 81:5849-5852; Acsadi et al, (1991) Nature 352:815-818; microprojectile bombardment (Williams et al. (1991) PNAS 88:2726-2730; liposomes of several types (see, e.g., Wang et al. (1987) PNAS 84:7851-7855; CaPO₄ (Dubensky et al. (1984) PNAS 81:7529-7533); DNA ligand (Wu et al. (1989) J. Biol. Chem. 264: 16985-16987; administration of nucleic acids alone (WO 90/11092); or administration of DNA linked to killed adenovirus (Curiel et al. (1992) Hum. Gene Ther. 3:147-154); via polycation compounds such as polylysine, utilizing receptor specific ligands; as well as with psoralen inactivated viruses such as Sendai or Adenovirus, by electroporation or by pressure-mediated delivery.

In a nonlimiting exemplary example, once a cancerous lesion is located, the vector formulation may be directly injected once or several times in several different locations within the body of the tumor. Alternatively, or additionally, arteries or blood vessels, which serve a tumor, may be identified and the vector injected into such blood vessel, in order to deliver the vector directly into the tumor. A tumor that has a necrotic center may be aspirated, and the vector injected directly into the now empty center of the tumor. The viral vector may be directly administered to the surface of the tumor, for example, by application of a topical pharmaceutical composition containing the viral vector. The vector may alternatively or additionally be administered by direct injection by other clinically acceptable means such as by various forms of catheter that can be introduced into the patient with minimal discomfort, followed by injection or release of the vector in conjunction with operations made possible by the catheter, such as multiple injection, introduction of radioactive seeds, tissue disruption and other means known to those skilled in the art. When the viral vector formulations are administered directly to the tumor or to the site of a resected tumor (where a tumor cell may still exist), from about 1×10⁶ to 1×10¹² viral vector or viral vector particles are administered either into the tumor or in the wall of the resection cavity at a number of sites ranging from about 1 to about 50 injection sites with a total volume injected of about 100 μl to about 5000 μl. The total intravenous dose of the viral vector can range from about 1×10⁷ to about 1×10¹² viral vectors or viral vector particles.

After the vector administration is completed, the prodrug formulation is administered. Administration can be oral or intravenous, depending on the prodrug. For orally administered prodrugs such as valacyclovir, dosing starts at about 1 days to about 3 days after viral vector administration at a dose between about 0.5 grams and about 2 grams orally about 1 to about 3 times a day for about 2 days to about 14 days. Certain patients, such as those with impaired renal function, may receive a modified dose schedule such as about 1.5 grams orally three times a day, or about 1.5 grams twice a day. Other prodrugs such as ganciclovir and acyclovir. Ganciclovir is administered intravenously at about 0.5 mg/kg to about 10 mg/kg up to twice daily for between about 5 days and about 14 days. Acyclovir is administered at about 5 mg/kg to about 20 mg/kg as frequently as every 8 hours for between about 5 days and about 14 days.

B. Administration of an ATR Inhibitor

ATRi treatment can be administered before, during, or after GMCI treatment. Details of the dosing of the ATRi, including the route of administration and dosage levels depend on the properties of the specific agent. These are typically characterized by balancing commonly used metrics of clinical efficacy (e.g., tumor shrinkage, survival, time to disease progression, improvements in symptoms) with side effects associated with administration of the drugs. By a “therapeutically effective amount” of the compound or formulation useful in the treatment methods according to the disclosure is meant a sufficient amount of the compound, composition, or formulation to treat or prevent a disease or disorder ameliorated by a ATRi inhibitor at a reasonable benefit/risk ratio applicable to any medical treatment. It will be understood, however, that the total daily usage of the compounds, formulations, and compositions useful in the treatment methods according to the disclosure will be decided by the attending physician within the scope of sound medical judgment. The specific therapeutically effective dose level for any particular patient will depend upon a variety of factors including the disorder being treated and the severity of the disorder; activity of the specific compound employed; the specific composition, formulation, and/or compound being administered, the age, body weight, general health, sex and diet of the patient; the time of administration, route of administration, and rate of excretion of the specific compounds, composition, and/or formulations employed; the duration of the treatment; compositions, compounds, or formulations used in combination or coincidental with others employed; and like factors well known in the medical arts. For example, it is well within the skill of the art to start doses of the compound at levels lower than those required to achieve the desired therapeutic effect and to gradually increase the dosage until the desired effect is achieved.

Administration of ATRi agents may be initiated at high dose levels to provide optimal efficacy. However, if dosing of an ATRi agent is associated with toxicity, gradually decrease the dosage of the agent may decrease associated toxicities, but also decrease efficacy. When a patient is administered a combination of an ATR inhibitor with GMCI, lower doses of the ATR inhibitor can be used while achieving optimal efficacy (such as amount or duration of tumor response, or increase in survival of the cancer patient), with the toxicity of profile of the combination therapy being improved.

### IV. Additional Associated Treatments

The combination therapy according to the disclosure can be used along with other cancer treatments, including, but not limited to standard cancer treatment modalities such as resectional surgery, radiation therapy, chemotherapy, and immune modulating therapies such as anti-checkpoint protein antibodies. The use of these additional therapies does not reduce the synergistic effect of the combination treatment.

### V. Cancers to be Treated

Cancer that can be treated by the method according to the disclosure include solid tumors, liquid tumors, and metastases, and micrometastases. Tumors such as, but not limited to, hyperplastic or neoplastic disease, such as a carcinoma, sarcoma, or mixed type cancer, including breast, colorectal, endometrial, gastric, prostate or brain, mesothelioma, ovarian, lung or pancreatic cancer can be targeted for therapy using the present method.

### VI. Monitoring of Treatment

Tumor cell death in vitro experiments can be measured using established methods such as assays for cell viability assays using agents such as MTT, MTS, or Alomar Blue (Speranza et al. 2017) and/or by standard clinical pathology methods of observing cell death by the analysis of necrosis in treated tissues.

Tumor size can be monitored using standard radiographic methods such as Magnetic Resonance Imaging (MRI) or Computed Tomography scan (CT Scan). Changes in tumor size are assessed using standard clinical criteria such as Response Evaluation Criteria In Solid Tumors (RECIST) (Therasse et al. 2000; Eisenhauer et al. 2009) or Immune Related Response Criteria (irRC) (Hodi et al. 2014, 2016) or Response Assessment in Neuro-oncology, or RANO Criteria (Okada et al. 2015).

### VII. Synergistic Effects of the Combined Therapy

Unexpectedly improved and surprising anti-cancer results were obtained when GMCI and DDRI therapies are combined. The cytotoxic activity in tumor cells treated with the combination therapy is greater than the cytotoxic activity found in cells treated with only GMCI or DDRI and is greater than the additive effect of both. Further, the cytotoxicity measured when using the combination is more than the cytotoxicity found when using GMCI or DDRI, alone or added together.

The combination theory results in more rapid killing of cancer cells and tumor shrinkage than was found when either therapy, alone, was used. Tumor responses to standard protocols (RECIST, irRC, RANO Criteria) occurred with more frequency in patients treated with the combination therapy, than in patients treated with only GMCI or an ATR inhibitor, alone. Also, tumor responses that occur in patients treated the combination of GMCI and an ATRi were greater and more durable than in patients treated with either agent alone.

When a patient is administered a combination of an ATR inhibitor with GMCI, lower doses of the ATRi can be used while achieving optimal efficacy (such as amount or duration of tumor response, or increase in survival of the cancer patient), with the toxicity of profile of the combination therapy being improved.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### GMCI in Combination with ATR Inhibitor for Treatment of Brain Tumors

As per current standard of care, brain cancer patients typically undergo surgical removal of the tumor in followed by treatment with radiation therapy and temozolomide. Radiation therapy dosing is between about 70 Gy and about 80 Gy of radiation over a period of about 3 weeks to about 8 weeks.

The AdV-tk vector is injected into the resection cavity post-surgery. Between about 1×10¹⁰ and about 1×10¹² vector particles are delivered in a total volume in the range of 0.5 ml and 2 ml over about 5 to about 50 sites within the surgical cavity.

The patient then receives a course of prodrug, valacyclovir. Prodrug treatment begins about 1 day to about 3 days after vector administration at a dose of approximately 2 g orally 3 times a day for 14 days. When a patient is unable to take the oral prodrug for any reason, intravenous acyclovir at 10 mg/kg tid is substituted.

Administration of the DDRI drug AZD6738 is initiated with the first dose about 1 day to about 7 days before surgery and AdV-tk injection. Continuous or intermittent dosing continues during the course of radiation therapy and for about 2 weeks after the radiation course ends. AZD6738 is dosed orally at between 0.1 mg/kg and 30 mg/kg per day.

Clinical patient outcomes are monitored using standard methodology, including, but not limited to, at least one of tumor response, disease progression, quality of life, blood chemistry, immune system status, general wellness, and/or survival.

The patients receiving the combination treatment have improved outcomes when compared to patents with similar disease characteristics that receive current standard of care or either single agent alone. Improvements in outcomes include, but are not limited to, one or more of improved survival time post-treatment, increased time to disease recurrence, and/or a better quality of life.

### Example 2

### GMCI in Combination with an ATR Inhibitor for Treatment of Prostate Cancer

As per current standard of care, prostate cancer patients commonly undergo radiation therapy of the prostate. Radiation therapy dosing is between about 70 Gy and about 80 Gy of radiation over a period of about 3 weeks to about 8 weeks.

Three courses of GMCI are administered. he first course of GMCI is started about 1 week to about 5 weeks before the initiation of radiation therapy. The second GMCI course is started in the first 3 weeks of radiation therapy. The third course of GMCI is started about 5 weeks to about 8 weeks after the initiation of radiation therapy.

For each course the AdV-tk vectors are injected into the prostate. Between about 1×10¹⁰ and about 1×10¹² vector particles in a total volume of about 0.5 ml to about 2 ml are delivered over 4 sites within the prostate gland. For each course, after the injection of the vector, the patient receives a course of the prodrug, valacyclovir. Prodrug treatment begins about 1 day after vector administration at a dose of about 2 g orally 3 times a day for about 14 days. If a patient is unable to take the oral prodrug for any reason, intravenous acyclovir at 10 mg/kg patient weight is substituted.

Administration of the ATRi drug, AZD6738, is initiated with the first dose about 1 days before to about 14 days after the start of the first course of GMCI. Continuous or intermittent dosing continues during the radiation therapy course and for about 2 weeks after the radiation course ends. AZD6738 is dosed orally at between about 0.1 mg/kg and about 30 mg/kg per day.

Clinical patient outcomes are monitored using standard methodology, including, but not limited to, at least one of tumor response, disease progression, quality of life, blood chemistry, immune system status, general wellness, and/or survival.

The patients receiving the combination treatment have improved outcomes when compared to patents with similar disease characteristics that receive current standard of care or either single agent alone. Improvements in outcomes include, but are not limited to, one or more of improved survival time post-treatment, increased time to disease recurrence, and/or a better quality of life.

### Example 3

### GMCI in Combination with an ATRi Inhibitor for Treatment of Ovarian Cancer

As per current standard of care, ovarian cancer patients commonly undergo tumor debulking. After tumor removal, between about 1×10¹⁰ and about 1×10¹³ vector particles are administered intraperitoneally in a total volume of about 5 ml to about 500 ml. After vector administration, the patient receives a course of prodrug, valacyclovir. Prodrug treatment begins about 1 day after vector administration at a dose of about 2 g orally 3 times a day for about 14 days. If a patient is unable to take the oral prodrug for any reason, intravenous acyclovir at 10 mg/kg tid is substituted.

Administration of a ATRi drug such as VX-970 is initiated about 1 day to about 10 days before the initiation of GMCI. Continuous or intermittent dosing of VX-970 continues during the prodrug course, and continue for 1 week to 2 weeks after the prodrug course ends. VX-970 is dosed intravenously at between 0.1 mg/m² and 799 mg/m² per day in day 1, day 2, and day 9 in 21 day cycles. (Shapiro 2016, 10.1158/1538-7445.AM2016-CT012).

Clinical patient outcomes are monitored using standard methodology, including, but not limited to, at least one of tumor response, disease progression, quality of life, blood chemistry, immune system status, general wellness, and/or survival.

The patients receiving the combination treatment have improved outcomes when compared to patents with similar disease characteristics that receive current standard of care or either single agent alone. Improvements in outcome include, but are not limited to, improved survival time post-treatment, increased time to disease recurrence, and/or better quality of life.

### Example 4

### GMCI—ATR Inhibitor Combination

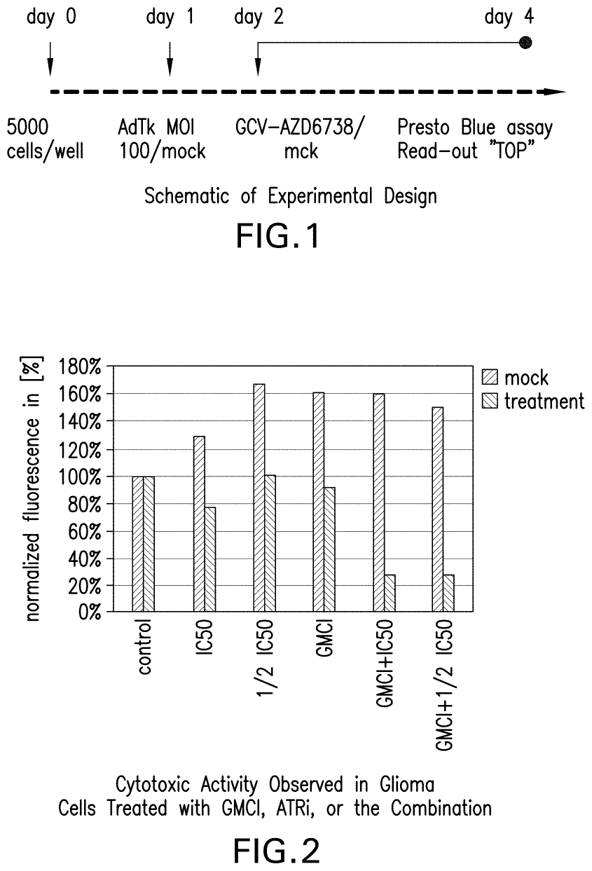

The combination of GMCI and ATRi (AZD6738) is examined for anti-cancer activity. A schematic of an experimental design to test the effect of combining of GMCI and ATR inhibitor combination on glioma cells is illustrated in FIG. 1. At the start of the study (Day 0), a glioma cell line (GL261; NCI-Frederick Cancer Research Tumor Repository, Frederick, Md.) is plated at a density of 5000 cells per well. For the samples on which GMCI is administered, on the following day (Day 1) AdV-tk vector is added at a concentration of approximately 5×104 vp/ml (100 MOI). Starting on Day 2, to the samples being treated with GMCI, ganciclovir is added to a concentration of 5 μg/ml. In samples exposed to ATRi, starting at Day 2 AZD6738 was added at a concentration to either the IC50 concentration (1 μM) of the drug, or ½ the IC₅₀ concentration (0.5 μM) of the drug, either in combination with GMCI or alone, as indicated. At Day 4, cell viability is assayed by the addition of a vial dye such as Presto Blue assay (Life Technologies) and quantified by fluorescence spectrometry. The viability of cells exposed to various regimens (GMCI, ATRi, the combination of GMCI and ATRi (AZD6738), or neither treatment).

After treatment of glioma cells as described, significant differences in the viability of the cancer cells were observed. Observed fluorescence, which is a measurement of viability in this assay, is normalized to samples that received no treatment and plotted in FIG. 2. ATRi is observed to have cytotoxic activity when dosed at either its IC₅₀ concentration or half of its IC₅₀ concentration. GMCI also provides a decrease in cell activity indicating cytotoxic activity as previously observed.

When ATRi and GMCI treatments were combined, there was a large, unanticipated decrease in cell viability at either the IC₅₀ concentration of ATRi or half the IC₅₀ concentration of ATRi. The decrease was greater than any observed with either agent by itself.

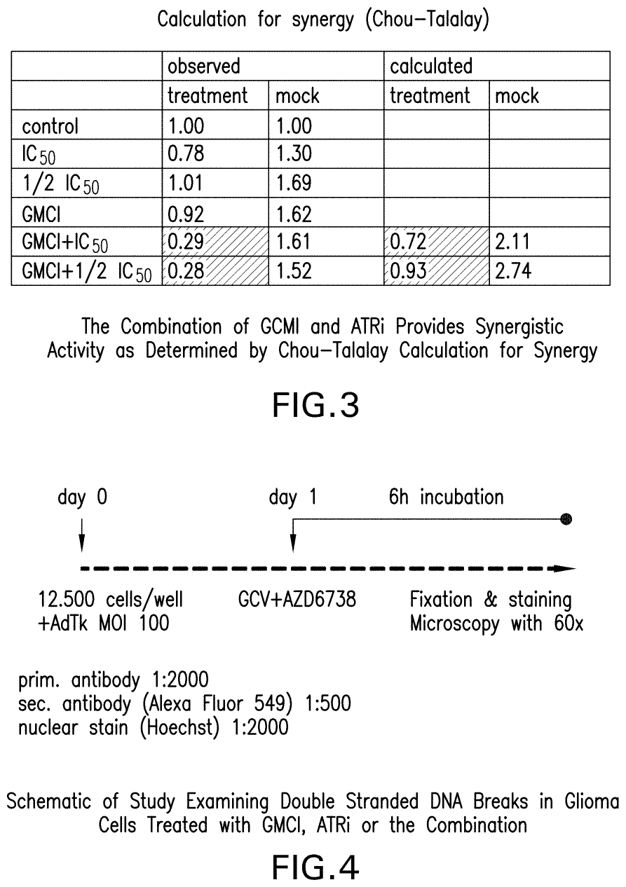

FIG. 3 shows the statistical analysis demonstrating synergy between GMCI and ATRi in the reduction of viability of cancer cells. Synergism was analyzed by the Combination Index (CI) calculation with the formula of Chou-Talalay (Chou 2010; Ting-Chao Chou 1984). The results are shown in FIG. 2. CI values above 1.00 indicate antagonistic effect of combination agents, a CI of 1.00 indicates an additive effect, and CI values below 1.00 indicate synergistic effect. The calculated CI for GMCI with the ATRi at IC50 concentration is 0.72, and GMCI combined with ATRi at ½ IC₅₀ concentration is 0.93, indicating synergism.

In another experiment, the frequency of double-stranded DNA breaks in glioma cells treated with GMCI, ATRi, or the combination. The formation of double-stranded DNA breaks was examined by staining the cells for H2AX-Ser139 as previously performed (Speranza et al. 2017).

At the start of the study (Day 0), glioma cell line (GL261; NCI-Frederick Cancer Research Tumor Repository, Frederick, Md.) is plated at a density of 12,500 cells per well of a 24 well plate containing glass coverslips. For the samples to which GMCI is administered, AdV-tk vector is added at a concentration of approximately 1.25×10⁶ vp/ml (100 MOI). Starting on Day 1, to the samples being treated with GMCI, ganciclovir is added to a concentration of 5 μg/ml. In samples exposed to ATRi, starting at Day 1 AZD6738 was added at a concentration to the IC50 concentration of the drug. either in combination with GMCI or alone, as indicated. At a timepoint 6 hours after addition of the ATRi and/or GCV, the cells were fixed in 4% paraformaldehyde in phosphate buffered saline. After blocking with 5% donkey serum/0.5% Tween 20, 0.02% TX100/PBS for 1 hour at room temperature (RT), cells were washed 3 times with washing buffer (0.5% Tween 20, 0.02% TX100/PBS) and stained with H2AX Ser139 antibody (1:100). The next day the samples were washed and incubated for 2 hours with a fluorescently-tagged secondary antibody plus Hoechst 33342. Images were captured with a Zeiss LSM710 confocal microscope.

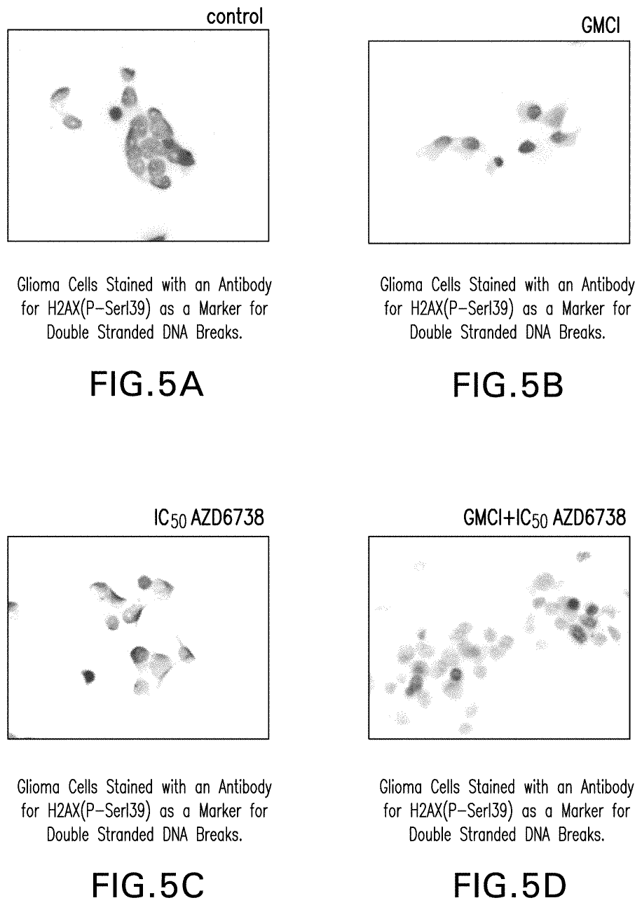

FIGS. 5A-5D show the results of a study examining the frequency of double-stranded DNA breaks in glioma cell treated the GMCI, ATRi, or the combination for the two treatment regimens. Double-stranded DNA breaks occur in treatment with GMCI and/or with ATRi (AZD6738) (FIGS. 5B and 5D). H2AX Ser139 (FIGS. 5C and 5D) quantification of glioblastoma cell lines (GL261Luc2 and CT-2ALuc) after AdV-tk, GCV, and GMCI treatments compared with mock (FIG. 5A). All samples were stained with Hoescht dye which marks DNA containing regions.

Confocal microscopic images of CT-2Aluc cells after AdV-tk, GCV, and GMCI treatments showing nuclear staining with an antibody specific for phospho-histone H2AX (Ser139) (anti-H2AX Ser139 (Product #9718, Cell Signal Technologies) in green and Hoechst in blue. Untreated cells show very little staining for H2AX(Ser139), whereas GMCI, ATRi and combination samples shows a large increase in H2AX staining.

### EQUIVALENTS

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and, described herein will become apparent to those skilled in the art from the foregoing description and accompanying drawings. Such modifications are intended to fall within the scope of the appended claims.

1. A method of treating a cancer in a subject, comprising treating the subject with a combination of gene-mediated cytotoxic immunotherapy and an ATR inhibitor which is a DDRI.

2. The method of claim 1, wherein GMCI comprises:
i.) administering a viral vector encoding thymidine kinase or cytosine deaminase to the mammal with a tumor or to a tumor resection site in the mammal; and
ii.) administering a prodrug to the mammal, the prodrug being activated by thymidine kinase or cytosine deaminase.

3. The method of claim 1, wherein the vector is an adenovirus, an adeno-associated virus (AAV), a lentivirus, a retrovirus, a herpes virus, a New Castle Disease Virus, a coxsackievirus, or a vaccinia virus.

4. The method of claim 2, wherein the vector is replication-incompetent or replication deficient.

5. The method of claim 2, wherein the prodrug comprises ganciclovir, acyclovir, valacyclovir, valgancyclovir, famiciclovir, or an analog thereof.

6. The method of claim 2, wherein the prodrug comprises de 5-flurocytosine or an analog thereof.

7. The method of claim 1, wherein ATRi administration is before, during, or after GMCI administration.

8. The method of claim 1, wherein the ATR inhibitor is an inhibitor of ataxia-telangiectasia mutated kinase and Rad3-related kinase

9. The method of claim 1, wherein the ATR inhibitor is BAY1895344, Schisandrin B, NU6027, NVP-BEZ235, VX-803, VX-970(M6620), VE-821, VE-822, AZ20, or AZD6738

10. The method of claim 1, further comprising administering radiotherapy and/or chemotherapy to, and/or performing surgery on, the mammal before, during, or following GMCI and/or administering the ATR inhibitor.
